(19) United States
(12) Patent Application Publication
(10) Pub. No.: US 2010/0106242 A1
(43) Pub. Date: Apr. 29, 2010

(54) STENT AND METHOD FOR MAKING A STENT

(75) Inventors: Arzu M. Ozkan, Mountain View, CA (US); Chad Joseph Abunassar, San Francisco, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 12/257,327

(22) Filed: Oct. 23, 2008

(51) Int. Cl. A61F 2/06; A61B 19/00

(52) U.S. Cl. 623/1.42; 623/1.15; 623/1.44; 128/898

(57) ABSTRACT

A stent includes a body, a layer of therapeutic agent over at least a section of the body, and a sealant layer over the layer of therapeutic agent. The sealant layer includes a through hole that allows release of the therapeutic agent of the therapeutic agent layer through the through hole when the stent is deployed in a blood vessel.

### FIELD OF THE INVENTION

This invention relates to a stent and a method for making a stent.

### BACKGROUND OF THE INVENTION

Minimally invasive surgical procedures, such as percutaneous transluminal coronary angioplasty (PTCA), have become increasingly common. A PTCA procedure involves the insertion of a catheter into a coronary artery to position an angioplasty balloon at the site of a stenotic lesion that is at least partially blocking the coronary artery. The balloon is then inflated to compress the stenosis and to widen the lumen in order to allow an efficient flow of blood through the coronary artery.

Following PTCA and other stenotic treatment procedures, a significant number of patients experience restenosis or other vascular blockage problems. These problems are prone to arise at the site of the former stenosis.

In order to help avoid restenosis and other similar problems, a stent may be implanted into the vessel at the site of the former stenosis with a stent delivery catheter. A stent is a tubular structure which is delivered to the site of the former stenosis or lesion and expanded to compress against vessel walls thereat, again with a balloon. The structure of the stent promotes maintenance of an open vessel lumen. The stent can be implanted in conjunction with the angioplasty.

FIG. 1 illustrates a stent 10 formed from a plurality of struts 12. The plurality of struts 12 are radially expandable and interconnected by connecting elements 14 that are disposed between adjacent struts 12, leaving lateral openings or gaps 16 between adjacent struts 12. The struts 12 and connecting elements 14 define a tubular stent body having an outer, tissue-contacting surface and an inner surface.

A stent can also be used to provide for local delivery of a drug (i.e., a therapeutic agent). For example, radiotherapy and drug delivery treatments applied to the site of the former stenosis following angioplasty have been found to aid in the healing process and to reduce significantly the risk of restenosis and other similar problems. Local delivery of drugs is often preferred over systemic delivery of drugs, particularly where high systemic doses are necessary to achieve an effect at a particular site. High systemic doses of drugs can often create adverse effects. One proposed method of local delivery is to coat the surface of a stent with a drug.

Spray coating is commonly used to apply a layer of coating to a stent. A spray coating system typically includes a spray nozzle and a pump that supplies a coating substance from a reservoir to the spray nozzle. The coating substance is ejected through the nozzle and applied to the surface of the stent.

### SUMMARY OF THE INVENTION

Recent studies indicate that the structure of a deployed stent may aggravate restenosis and other similar problems. FIG. 2 provides an example. In FIG. 2, a strut 12 of a stent 10, which is deployed in a blood vessel 20, is shown contacting the wall of the blood vessel 20. The blood flows from the left of the figure to the right, as indicated by an arrow 22. The stent strut 12 impedes and disrupts blood flow, generating flow recirculation or stagnation in the area 24 “behind” the strut 12. In an area of flow recirculation or stagnation and low wall shear stress, restenosis tends to occur, and the severity of restenosis tends to be greater. Additionally, flow recirculation or stagnation, combined with the inflammatory response prompted by local injury generated by stent deployment, may potentially lead to the formation of acute, sub-acute, or potential late stent thrombosis. It may also lead to platelet deposition, aggregation, and accumulation.

The present invention relates to a stent that may be used to alleviate the above-discussed problems. In one embodiment of the invention, a stent can release one or more drugs at specified rates in the areas of the stent that are susceptible to blood recirculation or stagnation. In general, a stent of the present invention may be made to release different drugs or different combinations of drugs at different rates in different areas of the stent. Additionally or alternatively, the drugs in different areas of the stent may be released in different chronological sequences. In this respect, this stent of the present invention is advantageous over a spray-coated stent, because the coating of a spray-coated stent is uniform. In other words, a spray-coated stent has the same types of drugs coated throughout its surface, and the drugs have the same release rate when it is deployed in a blood vessel. It is very difficult, if not impossible, to vary the types of coated drugs, or to vary the release rate of the coated drugs, from one area of the stent to another area. Additionally, as discussed below, a stent of the present invention can be made efficiently and cost-effectively, and can be adapted to the specific needs of patients efficiently and cost-effectively.

In accordance with one aspect of the present invention, a stent includes a body, a layer of therapeutic agent over at least a section of the body, and a sealant layer over the layer of therapeutic agent.

According to a preferred embodiment of this aspect of the invention, the sealant layer includes a through hole that allows release of the therapeutic agent of the therapeutic agent layer through the sealant layer.

According to another preferred embodiment of this aspect of the invention, the sealant layer includes a first section including at least one through hole, and a second section including at least one through hole. The first and second sections are of the same size, and the total area of the at least one through hole of the first section is greater than the total area of the at least one through hole of the second section.

According to still another preferred embodiment of this aspect of the invention, the first section borders an area of blood flow recirculation or stagnation when the stent is deployed in a blood vessel.

According to yet another preferred embodiment of this aspect of the invention, the sealant layer includes a third section including at least one through hole, and the total area of the at least one through hole of the third section is different from the total area of the at least one through hole of the first section and from the total area of the at least one through hole of the second section.

According to yet still another preferred embodiment of this aspect of the invention, the sealant layer is dissolvable when the stent is deployed in a blood vessel.

According to a further preferred embodiment of this aspect of the invention, the sealant layer includes a first section including a plurality of through holes, and a second section including a plurality of through holes. The first and second sections are of the same size, and the number of through holes of the first section is greater than the number of through holes of the second section.

According to a still further preferred embodiment of this aspect of the invention, the sealant layer is dissolvable when the stent is deployed in a blood vessel.

According to a yet further preferred embodiment of this aspect of the invention, the sealant layer includes a therapeutic agent, wherein the therapeutic agent of the therapeutic agent layer is different from the therapeutic agent of the sealant layer.

According to another preferred embodiment of this aspect of the invention, the sealant layer includes no therapeutic agent.

In accordance with another aspect of the present invention, a stent includes a body, a first layer of therapeutic agent over at least a section of the body, a first sealant layer over the first layer of therapeutic agent, a second layer of therapeutic agent over the first sealant layer, and a second sealant layer over the second layer of therapeutic agent.

According to one preferred embodiment of this aspect of the invention, the stent further includes a first through hole that extends through the second sealant layer to reach the second therapeutic agent layer to allow release of the therapeutic agent of the second therapeutic agent layer through the first through hole.

According to another preferred embodiment of this aspect of the invention, the stent further includes a second through hole that extends through the second sealant layer, the second therapeutic agent layer, and the first sealant layer to reach the first therapeutic agent layer to allow release of the therapeutic agent of the first therapeutic agent layer through the second through hole.

According to still another preferred embodiment of this aspect of the invention, the stent further includes a third through hole that extends through the second sealant layer and the second therapeutic agent layer to reach the first sealant layer.

According to yet another preferred embodiment of this aspect of the invention, the stent further includes a through hole that extends through the second sealant layer, the second therapeutic agent layer, and the first sealant layer to reach the first therapeutic agent layer to allow release of the therapeutic agent of the first therapeutic agent layer through the through hole.

According to yet still another preferred embodiment of this aspect of the invention, the stent further includes a first section that includes a first set of at least one hole extending through the second sealant layer to reach the second therapeutic agent layer and a second set of at least one hole extending through the second sealant layer, the second therapeutic agent layer, and the first sealant layer to reach the first therapeutic agent layer. The stent further includes a second section that includes a third set of at least one hole extending through the second sealant layer to reach the second therapeutic agent layer and a fourth set of at least one hole extending through the second sealant layer, the second therapeutic agent layer, and the first sealant layer to reach the first therapeutic agent layer.

According to a further preferred embodiment of this aspect of the invention, the first and second sections are of the same size, and the ratio of the total area of the first set of at least one hole over the total area of the second set of at least one hole is greater than the ratio of the total area of the third set of at least one hole over the total area of the fourth set of at least one hole.

According to a still further preferred embodiment of this aspect of the invention, the first section borders an area of blood flow recirculation or stagnation when the stent is deployed in a blood vessel.

According to a yet further preferred embodiment of this aspect of the invention, the second section borders an area of blood flow recirculation or stagnation when the stent is deployed in a blood vessel.

According to a yet still further preferred embodiment of this aspect of the invention, the first sealant layer is dissolvable when the stent is deployed in a blood vessel.

According to another preferred embodiment of this aspect of the invention, the second sealant layer is dissolvable when the stent is deployed in a blood vessel.

According to another preferred embodiment of this aspect of the invention, the first and second sections are of the same size, and the ratio of the number of holes in the first set over the number of holes in the second set is greater than the ratio of the number of holes in the third set over the number of holes in the fourth set.

According to still another preferred embodiment of this aspect of the invention, the first sealant layer includes a therapeutic agent.

According to yet another preferred embodiment of this aspect of the invention, the first sealant layer includes no therapeutic agent.

According to yet still another preferred embodiment of this aspect of the invention, the second sealant layer includes a therapeutic agent.

According to a further preferred embodiment of this aspect of the invention, the second sealant layer includes no therapeutic agent.

Still another aspect of the present invention is directed to a method of fabricating a stent comprising a body, a layer of therapeutic agent over at least a section of the body, and a sealant layer over the layer of therapeutic agent. The method includes the step of using a pulsed laser beam to drill a through hole through the sealant layer. The hole allows release of the therapeutic agent of the therapeutic agent layer through the through hole when the stent is deployed in a blood vessel. The pulsed laser beam has a pulse duration of less than one picosecond.

According to a preferred embodiment of this aspect of the invention, a wavelength of the pulsed beam is less than or equal to 800 nm.

According to another preferred embodiment of this aspect of the invention, a repetition rate of the laser is 10 to 100 kHz.

A further aspect of the present invention is directed to a method of fabricating a stent comprising a body, a first layer of therapeutic agent over at least a section of the body, a first sealant layer over the first layer of therapeutic agent, a second layer of therapeutic agent over the first sealant layer, and a second sealant layer over the second layer of therapeutic agent. The method includes the step of using a pulsed laser beam to drill a hole through at least one of the second sealant layer, the second layer of therapeutic agent, and the first sealant layer, wherein the pulsed laser beam has a pulse duration of less than one picosecond.

According to a preferred embodiment of this aspect of the invention, the hole extends through the second sealant layer to reach the second therapeutic agent layer.

According to another preferred embodiment of this aspect of the invention, the method further includes using a pulsed laser beam to drill another hole that extends through the second sealant layer, the second therapeutic agent layer, and the first sealant layer to reach the first therapeutic agent layer.

According to a further preferred embodiment of this aspect of the invention, the hole extends through the second sealant layer and the second therapeutic agent layer to reach the first sealant layer.

### BRIEF DESCRIPTION OF THE DRAWINGS

FIG. 1 is a partial perspective view of a stent

FIG. 2 illustrates an area of blood flow recirculation or stagnation caused by the structure of a stent.

FIG. 3 is a partial cross-section view of an embodiment of the present invention.

FIG. 4 is a partial cross-section view of another embodiment of the present invention.

FIG. 5 is a partial cross-section view of still embodiment of the present invention.

FIG. 6 is a partial cross-section view of a further embodiment of the present invention.

FIG. 7 illustrates a chirped pulse application process.

FIG. 8 illustrates the function and components of the amplifier illustrated in FIG. 7.

### DETAILED DESCRIPTION OF PREFERRED EMBODIMENTS

A preferred embodiment of the stent according to the present invention may have the configuration shown in FIG. 1. The stent 10 shown in FIG. 1 includes a plurality of struts 12 that are radially expandable. The struts 12 may be interconnected by connecting elements 14 that are disposed between adjacent struts 12, leaving lateral openings or gaps 16 between the adjacent struts 12. The struts 12 and connecting elements 14 define a tubular stent body having an outer, tissue-contacting surface and an inner surface.

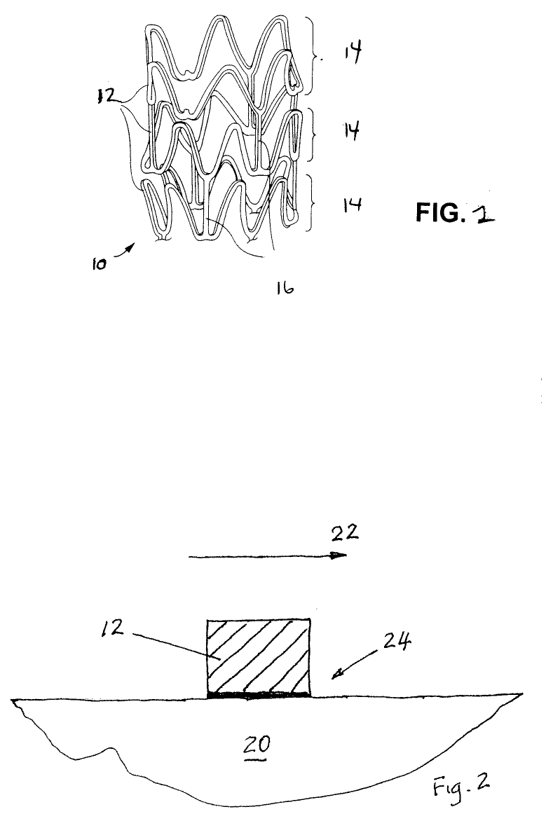

In the illustrated preferred embodiment of the present invention, a first area of the stent surface may have different drug release characteristics than a second area of the stent surface. The first area may be adjacent or may border the area 24 of blood flow recirculation or stagnation, as shown in FIG. 2, and therefore require certain drug release characteristics. And the second area may be remote from the area 24 of blood flow recirculation or stagnation and therefore require different drug release characteristics.

In the present invention, the different drug release characteristics may include one or more of the following: different types of released drugs, different rates of release for the drugs, and different chronological sequences of release for the drugs. In the preferred embodiment of the present invention, the different drug release characteristics are achieved by applying one or more alternating drug and sealant layers on the stent body and by drilling through holes in one or more drug and sealant layers.

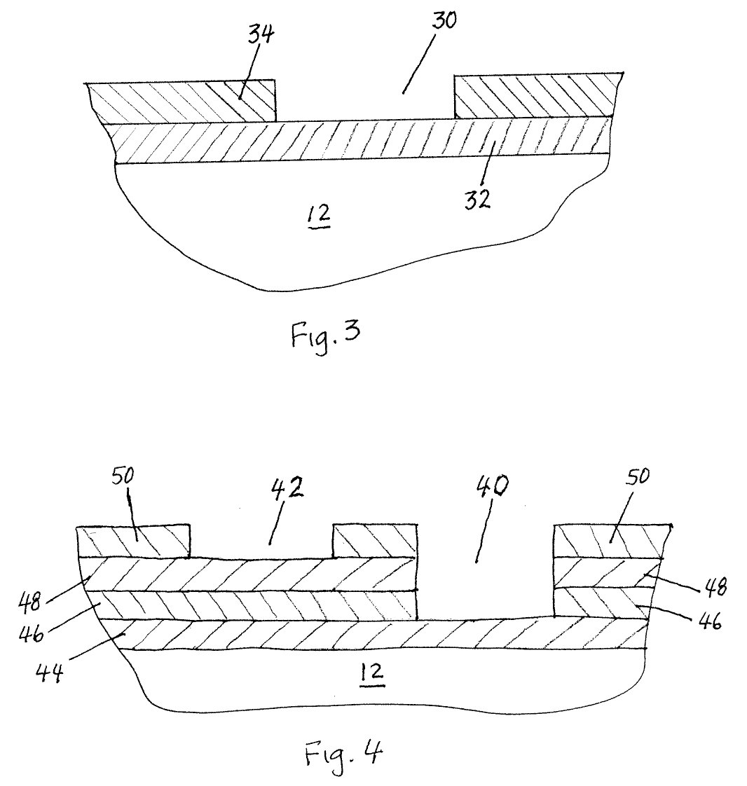

FIG. 3 illustrates such a through hole 30. In FIG. 3, a drug layer 32 is coated on the stent 12, and then a sealant layer 34 is coated on the drug layer 32. The through hole 30 is drilled through the sealant layer 34 to reach the drug layer 32. The drug in the portion of the drug layer 32, which is exposed by the hole 30, can be released through the hole 30 into the blood stream when the stent 12 is deployed in a blood vessel. The drug in the portion of the drug layer 32, which is covered by the sealant layer 34, is not released (or is released at a lower rate if the sealant layer is permeable to some degree). Therefore, the release rate of the drug can be controlled by the area of the hole 30. In other words, a larger hole increases the drug's release rate, while a smaller hole decreases the drug's release rate. For a given area of the stent surface, the release rate of the drug can be varied by the number of holes 30 in the area and/or by the sizes of the holes 30 in the area.

In some embodiments of the present invention, the sealant layer 34 may contain a drug, which may be different from the drug in the drug layer 32. The drug in the sealant layer 34 may also be released to the blood stream, and the release rate may be different from the release rate of the drug in the drug layer 32.

FIG. 4 illustrates two additional through holes 40, 42 of the present invention. In FIG. 4, a first drug layer 44 is coated on the stent 12, and a first sealant layer 46 is coated on the first drug layer 44. Then a second drug layer 48 is coated on the first sealant layer 46, and a second sealant layer 50 is coated on the second drug layer 48.

The first 40 of the two through holes 40, 42 extends through the second sealant layer 50, the second drug layer 48, and the first sealant layer 46 to reach the first drug layer 44. The drug in the portion of the first drug layer 44, which is exposed by the first hole 40, can be released through the first hole 40 into the blood stream when the stent 12 is deployed in a blood vessel. The drug in the portion of the first drug layer 44, which is covered by the first sealant layer 46, is not released. Therefore, the release rate of the drug in the first drug layer 44 can be controlled by the size of the first hole 40. The second hole 42 extends through the second sealant layer 50 to reach the second drug layer 48. The drug in the portion of the second drug layer 48, which is exposed by the second hole 42, can be released through the second hole 42 into the blood stream when the stent 12 is deployed in a blood vessel. The drug in the portion of the second drug layer 48, which is covered by the second sealant layer 50, is not released. Therefore, the release rate of the drug in the second drug layer 48 can be controlled by the size of the second hole 42. The ratio of the release rates of the two drugs can be adjusted by the ratio of the areas of the two holes 40, 42.

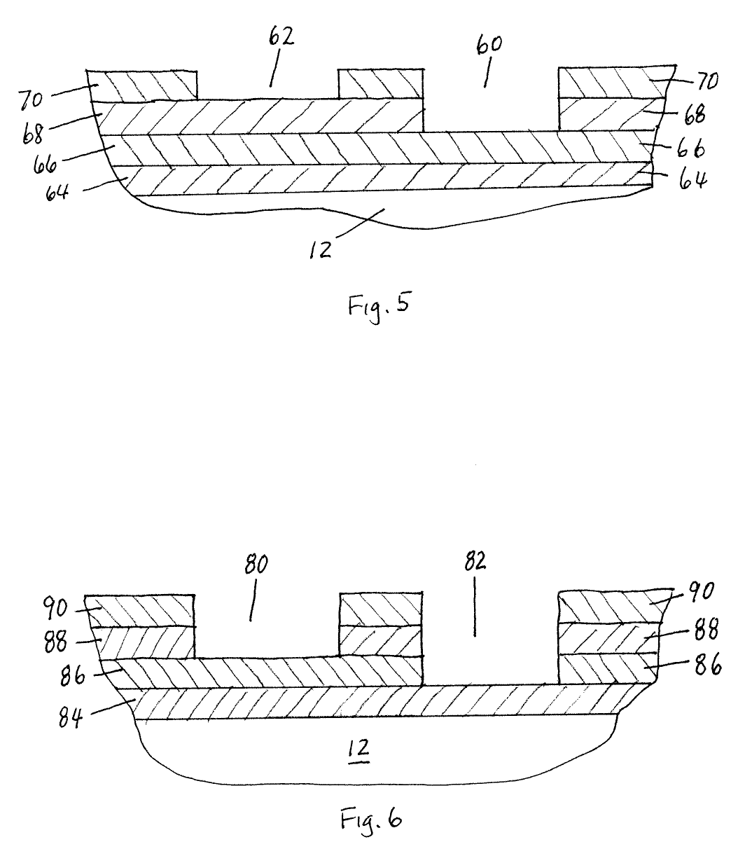

FIG. 5 illustrates two further through holes 60, 62 of the present invention. In FIG. 5, a first drug layer 64 is coated on the stent 12, and a first sealant layer 66 is coated on the first drug layer 64. Then a second drug layer 68 is coated on the first sealant layer 66, and a second sealant layer 70 is coated on the second drug layer 68.

The first 60 of the two through holes 60, 62 extends through the second sealant layer 70 and the second drug layer 68 to reach the first sealant layer 66. The second hole 62 extends through the second sealant layer 70 to reach the second drug layer 68. The drug in the portion of the second drug layer 68, which is exposed by the second hole 62, can be released through the second hole 62 into the blood stream when the stent 12 is deployed in a blood vessel. The first sealant layer 66 is dissolvable so that the drug in the first drug layer 64 is released only after the area of the first sealant layer 66 exposed by the first hole 60 has been dissolved after a period of time. In this way, the drug in the second drug layer 68 is released first, and the drug in the first drug layer 64 is released subsequently.

FIG. 6 illustrates two still further through holes 80, 82 of the present invention. In FIG. 6, a first drug layer 84 is coated on the stent 12, and a first sealant layer 86 is coated on the first drug layer 84. Then a second drug layer 88 is coated on the first sealant layer 86, and a second sealant layer 90 is coated on the second drug layer 88.

The first 80 of the two through holes 80, 82 extends through the second sealant layer 90 and the second drug layer 88 to reach the first sealant layer 86. The first sealant layer 86 is dissolvable so that the drug in the first drug layer 84 in the first hole 80 is released only after the area of the first sealant layer 86 exposed by the first hole 80 has been dissolved after a period of time. The second through holes 82 extends through the second sealant layer 90, the second drug layer 88, and the first sealant layer 86 to reach the first drug layer 84. The drug in the portion of the first drug layer 84, which is exposed by the second hole 82, can be released through the second hole 82 into the blood stream when the stent 12 is deployed in a blood vessel. In this way, the drug in the first drug layer 84 is first released at a lower rate only through the second hole 82, and is then released at a higher rate through both the first and second holes 80, 82.

Although FIGS. 3-6 show two and four alternating drug and sealant layers, it should be clear to one of ordinary skill in the art that a stent of the present invention may have six, eight or more alternating drug and sealant layers. And holes can be drilled reach any of the drug layers, and the sizes of the holes can be adjusted to control the release rates of the drugs. In some embodiments, the outer most layer may be a drug layer which is directly released into the blood stream.

The holes shown in FIGS. 3-6 can be used to control the release of different drugs or different combinations of drugs at different rates in different areas of the stent. Additionally or alternatively, they can be used to release the drugs in different areas of the stent in different chronological sequences. For example, if it is desirable to release proportionally more drug in a first area of the stent surface adjacent or bordering the area 24 of blood flow recirculation or stagnation (FIG. 2) and to release less drug in a second area of the stent surface remote from the area 24 of blood flow recirculation or stagnation, then the first area may have more and/or larger holes 30 (FIG. 3) per unit area than the second area. For another example, if it is desirable to release a first drug in the first area and a second drug in the second area, then the holes 40, 42 shown in FIG. 4 may be used for this purpose, with the holes like the first hole 40 in the first area and the holes like the second hole 42 in the second area, for example. For a further example, if it is desirable to release two drugs in sequence, the holes 60, 62 in FIG. 5 with a dissolvable first sealant layer 66 may be used.

Each of the through holes described above may be placed at any suitable location. For example, if a stent strut has a generally rectangular cross-section, a through hole may be placed on any of the four surfaces to facilitate the appropriate delivery of the drugs. The number of the holes and the types of drugs may be selected based on anticipated fluid dynamics including blood flow velocity and recirculation.

Each of the above-described drug and sealant layers may have a wide range of thickness. For example, in some cases, the thickness of a layer may be between 1 μm to 2 μm. In some other cases, the thickness of a layer may be in the range of 0.1 μm to 100 μm, or 0.5 μm to 50 μm.

A preferred method of drilling the above-described holes is to use pulsed lasers with ultrashort pulse widths, i.e., pulse widths that are in the femtosecond range (less than one picosecond). “Pulse width” refers to the duration of an optical pulse versus time. The duration can be defined in more than one way. Specifically, the pulse duration can be defined as the full width at half maximum (FWHM) of the optical power versus time.

In an embodiment of the present invention, the holes can be drilled with nanometer accuracy with a femtosecond pulsed laser operating at 800 nm fundamental wavelength. If the femtosecond pulsed laser is operating at harmonic wavelengths such as 400 nm and/or 266 nm, then because of optical diffraction limited spot size, the smallest achievable spot size is even smaller than what is predicted by the diffraction limit. Spots as small as 80 nm have been reported with commercially available femtosecond amplifier systems. With this precision, drilling a 100-500 nm size hole is possible with femtosecond pulsed lasers. If the femtosecond laser is also operating at less than 10 femtoseconds, then even smaller features (less than 100 nm) are achievable. If a femtosecond laser is operating at a high repetition rate such as 10 to 100 kHz, the drilling process becomes even more manufacturing transparent.

It is not viable to drill holes in a coating layer with a thickness in the range of 1 to 2 μm or 1 to 4 μm using lasers with nanosecond pulse widths because of the heat affected zone and chemical and mechanical degradation. Longer-pulse lasers remove material from a surface principally through a thermal mechanism. The laser energy that is absorbed results in a temperature increase at and near the absorption site. As the temperature increases to the melting or boiling point, material is removed by conventional melting or vaporization. Depending on the pulse duration of the laser, the temperature rise in the irradiated zone may be very fast, resulting in thermal ablation and shock. An advantage of ultrashort-pulse lasers over longer-pulse lasers is that the ultrashort-pulse laser deposits its energy so fast that is does not interact with the plume of vaporized material, which would distort and bend the incoming beam and produce a rough-edged cut.

A heat affected zone is a portion of the target substrate that is not removed, but is still heated by the beam. The heating may be due to exposure of the substrate from a section of the beam with an intensity that is not great enough to remove substrate material through either a thermal or nonthermal mechanism. For example, the portions of a beam near its edges may not have an intensity sufficiently high to induce formation of a plasma. Most beams have an uneven or nonuniform beam intensity profile, for example, a Gaussian beam profile.

A heat affected zone in a target substrate is undesirable for a number of reasons. In both metals and polymers, heat can cause thermal distortion and roughness at the machined surface. Polymers are particularly sensitive to heat. The heat can cause chemical degradation that can affect the mechanical properties and degradation rate.

Additionally, heat can modify molecular structure of a polymer, such as degree of crystallinity and polymer chain alignment. Mechanical properties are highly dependent on molecular structure. For example, a high degree of crystallinity and/or polymer chain alignment is associated with a stiff, high modulus material. Heating a polymer above its melting point can result in an undesirable increase or decrease in crystallinity once the polymer resolidifies. Melting a polymer may also result in a loss of polymer chain alignment, which can adversely affect mechanical properties. In addition, since heat from the laser modifies the properties of the substrate locally, the mechanical properties may be spatially nonuniform. Such nonuniformity may lead to mechanical instabilities such as cracking.

Unlike long-pulse lasers, ultrashort-pulse lasers allow material removal by a nonthermal mechanism. Extremely precise and rapid machining can be achieved with minimal thermal ablation and shock. The nonthermal mechanism involves optical breakdown in the target material which results in material removal. Optical breakdown tends to occur at a certain threshold intensity of laser radiation that is material dependent. Specifically each material has its own laser-induced optical breakdown threshold which characterizes the intensity required to ablate the material at a particular pulse width. During optical breakdown of material, a very high free electron density, i.e., plasma, is produced. The plasma can be produced through mechanisms such as multiphoton absorption and avalanche ionization.

The rate of laser drilling is an important factor in any manufacturing process. Increasing or maximizing process throughput can be accomplished by adjusting relevant process parameters. The repetition rate of a laser pulse is directly related to the rate of cutting or material removal from a construct. Thus, increasing the repetition rate allows increase of the scan rate of the laser across a substrate resulting in an increase in process throughput.

Femtosecond pulsed lasers typically used for fabricating implantable medical devices have a repetition rate of between 1 and 5 kHz. This range limits the rate that a device can be machined. Embodiments of the present invention include using femtosecond pulsed lasers with a repetition rates greater than 5 kHz. In particular, some embodiments include laser drilling with repetition rates between 5 and 10 kHz. Additional embodiments can include repetition rates greater than 10 kHz and up to 100 kHz.

Embodiments of the femtosecond pulsed lasers have pulse widths less than 10-12 seconds, less than 500 fs, 100-500 fs, 80-100 fs, 10-80 fs, or less than 10 fs. The energy per pulse and fluence of the laser is high enough to drill materials such as polymers, metals, and ceramics. The average power per pulse of a beam can be 0.01-4 W, or more narrowly 0.5-2 W. The peak power per pulse of a beam can be 12.5-5000 MW, or more narrowly 6.25-2500 MW.

An exemplary beam can have a wavelength of 800 nm and a power of 1.4 W. The energy per pulse for this beam in a 5-10 kHz repetition rate range can have a range of 140-280 μJ with a fluence of 178-357 mJ/cm² based on a 10 micron spot size. The peak power for this beam for a 500 fs pulse width is 280-560 MW. The peak power for a 100 fs pulse width is 1400-2800 MW. The peak power for an 80 fs pulse width is 3500-1750 MW. The peak power for a 10 fs pulse width is 14000-280000 MW.

In such embodiments, the throughput of a stent laser machining process can be increased significantly by increasing repetition rate from the 1-5 kHz range to the ranges of the of present invention. In particular, the repetition rates of the present invention can result in an increase in throughput by factors of two to four, or more over a 1-5 kHz repetition rate.

In an embodiment of the present invention, the laser system used to drill the holes may include an active medium within a laser cavity. Generally, an active or gain medium is a material that includes a collection of atoms or molecules that are stimulated to a population inversion which can emit electromagnetic radiation in a stimulated emission. The active medium can be positioned between highly reflective mirrors that reflect a laser pulse between the mirrors. A power source supplies energy or pumps the active medium so that the active medium can amplify the intensity of light that passes through it to produce a laser beam for machining.

A laser may be pumped in a number of ways, for example, optically, electrically, or chemically. Optical pumping may use either continuous or pulsed light emitted by a powerful lamp or a laser beam. Diode pumping is one type of optical pumping. A laser diode is a semiconductor laser in which the gain or amplification is generated by an electrical current flowing through a p-n junction. Laser diode pumping can be desirable since efficient and high-power diode lasers have been developed and are widely available in many wavelengths.

Amplification of ultrashort optical pulses, e.g., femtosecond pulses, in a gain medium to pulse energies needed for laser machining can create optical peak intensities that can result in pulse distortion or even damage of the gain medium. This can be effectively prevented by employing chirped-pulse amplification (CPA) in which the pulse intensity is reduced before amplification. In CPA, an ultrashort, low energy pulse is stretched temporally to a longer pulse width. The stretched or long, low energy pulse is amplified, increasing the energy of the pulse. The stretched or long, high energy pulse is then temporally compressed to an ultrashort, high energy pulse, for example, a femtosecond pulse.

FIG. 7 illustrates the steps and basic components of a CPA process. A seed laser 100, an ultrashort pulse oscillator, generates a seed pulse or an ultrashort, low energy pulse 102. Pulse 102 is chirped and temporally stretched to a much longer duration to produce a stretched, low energy pulse 106. Pulse 102 is stretched by means of a stretcher 104, which is a strongly dispersive element, e.g., a grating pair or a long fiber. The peak power is reduced to a level where the above mentioned detrimental effects in the gain medium are avoided upon amplification of pulse 106. Pulse 106 is then injected into an amplifier 108 to produce a stretched, high energy pulse 112. Amplifier 108 includes an active medium which is pumped by a pump laser 110. Pulse 112 is then compressed to an ultrashort, high energy pulse 116 by a dispersive compressor 114, i.e., an element with opposite dispersion (typically a grating pair), which removes the chirp and temporally compresses the pulses to a duration similar to the input pulse duration.

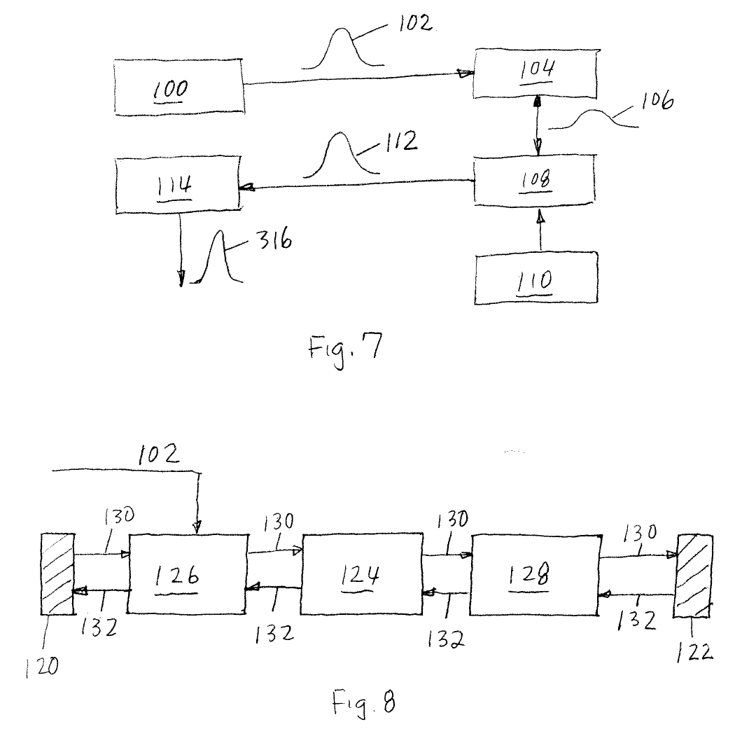

FIG. 8 illustrates the function and components of amplifier 108. Amplifier 108 includes highly reflective mirrors 120 and 122, active medium 124, injector 126, and ejector 128. Injector 126 samples and injects beam of stretched seed pulse 106 at a selected rate. A high speed driver (not shown) controls the repetition rate of seed pulses 106 injected into amplifier 108. In general, the sampling rate of the high speed driver is the repetition rate of the ultrafast, high energy pulse 116. The laser pulse from the pump laser must have a repetition rate at least that of the sampling rate of the high speed driver. A sampled pulse injected into amplifier 108 makes one or more round trips between mirrors 120 and 122 through active medium 124. The sampled pulses are amplified each round trip through active medium 124. Ejector 128 ejects pulse 116 from amplifier 108. The repetition rate of ejection is also controlled by the high speed driver.

Seed lasers for use with the present invention can include oscillators capable of generating a femtosecond pulse 102 with a repetition rate between 50 and 100 kHz with an output power of less than about 1.3 W.

Additionally, an embodiment of the present invention may have a high speed driver for use with amplifier 108 that is capable of sampling the stretched seed pulse 106 at a rate 5-10 kHz. This enables generation of an ultrafast, high energy pulse 116 with a repetition rate of 5-10 kHz. Additionally, the embodiment may include a pump laser that provides a beam with a repetition rate at least that of the sampling rate of the high speed driver. An exemplary pump laser is a 30 W Evolution Series laser from Coherent Inc. of Santa Clara, Calif. that generates a laser pulse of up to 15 kHz.

The stent 12 may be made from a variety of materials, such as a metal or a polymer. Representative examples of metallic material that may be used for fabricating a stent include, but are not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. A stainless steel tube or sheet may be Alloy type: 316L SS, Special Chemistry per ASTM F138-92 or ASTM F139-92 grade 2. Special Chemistry of type 316L per ASTM F138-92 or ASTM F139-92 Stainless Steel for Surgical Implants in weight percent. An exemplary weight percent may be as follows: Carbon (C) 0.03% max; Manganese (Mn): 2.00% max; Phosphorous (P): 0.025% max.; Sulphur (S): 0.010% max.; Silicon (Si): 0.75% max.; Chromium (Cr): 17.00-19.00%; Nickel (Ni): 13.00-15.50%; Molybdenum (Mo): 2.00-3.00%; Nitrogen (N): 0.10% max.; Copper (Cu): 0.50% max.; Iron (Fe): Balance.

Representative examples of polymers that may be used to fabricate a stent include, but are not limited to, poly(N-acetylglucosamine) (Chitin), Chitosan, poly(3-hydroxyvalerate), poly(lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose acetate, cellulose butyrate, cellulose acetate butyrate, cellophane, cellulose nitrate, cellulose propionate, cellulose ethers, and carboxymethyl cellulose. Additional representative examples of polymers that may be especially well suited for use in fabricating embodiments of implantable medical devices disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, poly(vinyl acetate), styrene-isobutylene-styrene triblock copolymers, and polyethylene glycol.

A stent may also be composed partially or completely of biodegradable, bioabsorbable or bioerodible materials. The terms biodegradable, bioabsorbable, and bioerodable, as well as degraded, eroded, and absorbed, are used interchangeably and refer to stent materials that are capable of being completely eroded or absorbed when exposed to bodily fluids such as blood and can be gradually resorbed, absorbed, and/or eliminated by the body.

Some metals are considered bioerodible since they tend to erode or corrode relatively rapidly when exposed to bodily fluids. Representative examples of biodegradable metals that may be used to fabricate stents may include, but are not limited to, magnesium, zinc, and iron. Polymers can be bioabsorbable, biodegradable, or bioerodable.

A stent made from a biodegradable material is intended to remain in the body for a duration of time until its intended function of, for example, maintaining vascular patency and/or drug delivery is accomplished. After the process of degradation, erosion, absorption, and/or resorption has been completed, no portion of the biodegradable stent, or a biodegradable portion of the stent will remain. In some embodiments, very negligible traces or residue may be left behind. The duration can be in a range from about a month to a few years. However, the duration is typically in a range from about one month to twelve months, or in some embodiments, six to twelve months.

The drug can include any substance capable of exerting a therapeutic or prophylactic effect for a patient. The drug may include small molecule drugs, peptides, proteins, oligonucleotides, and the like. The drug could be designed, for example, to inhibit the activity of vascular smooth muscle cells. It can be directed at inhibiting abnormal or inappropriate migration and/or proliferation of smooth muscle cells to inhibit restenosis.

Examples of drugs include antiproliferative substances such as actinomycin D, or derivatives and analogs thereof (manufactured by Sigma-Aldrich of Milwaukee, Wis., or COSMEGEN available from Merck). Synonyms of actinomycin D include dactinomycin, actinomycin IV, actinomycin T.sub.1, actinomycin X.sub.1, and actinomycin C.sub.1. The active agent can also fall under the genus of antineoplastic, anti-inflammatory, antiplatelet, anticoagulant, antifibrin, antithrombin, antimitotic, antibiotic, antiallergic and antioxidant substances. Examples of such antineoplastics and/or antimitotics include paclitaxel (e.g. TAXOL® by Bristol-Myers Squibb Co., Stamford, Conn.), docetaxel (e.g. Taxotere®, from Aventis S. A., Frankfurt, Germany) methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIIb/IIIa platelet membrane receptor antagonist antibody, recombinant hirudin, and thrombin inhibitors such as Angiomax™ (Biogen, Inc., Cambridge, Mass.). Examples of such cytostatic or antiproliferative agents include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents which may be appropriate include alpha-interferon, genetically engineered epithelial cells, tacrolimus, dexamethasone, and rapamycin and structural derivatives or functional analogs thereof, such as 40-O-(2-hydroxy)ethyl-rapamycin (known by the trade name of EVEROLIMUS available from Novartis), 40-O-(3-hydroxy)propyl-rapamycin, 40-O-[2-(2-hydroxy)ethoxy]ethyl-rapamycin, and 40-O-tetrazole-rapamycin.

A sealant layer of the present invention may include a deposited or electroplated thin metallic film made from any one or more of the metals described previously as being useable to make the stent. A sealant layer of the present invention may also include any one or more of the polymers described previously as being useable to make the stent. The sealant layer may range from minimally permeable to more permeable. Preferably, the maximum tolerable permeability of the sealant layer should be determined on the particular application. Examples of polymers suitable for use the sealant layer include poly(n-butyl methacrylate) (PBMA), a highly crystalline PLLA, poly(ethylene-co-vinyl acetate) (PEVA).

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A stent comprising:
a body;
a layer of therapeutic agent over at least a section of the body; and
a sealant layer over the layer of therapeutic agent.

2. The stent of claim 1, wherein the sealant layer includes a through hole that allows release of the therapeutic agent of the therapeutic agent layer through the through hole when the stent is deployed in a blood vessel.

3. The stent of claim 1, wherein the sealant layer includes
a first section including at least one through hole, and
a second section including at least one through hole, wherein the first and second sections are of the same size, and wherein the total area of the at least one through hole of the first section is greater than the total area of the at least one through hole of the second section.

4. The stent of claim 3, wherein the first section borders an area of blood flow recirculation or stagnation when the stent is deployed in a blood vessel.

5. The stent of claim 3, wherein the sealant layer includes a third section including at least one through hole, and wherein the total area of the at least one through hole of the third section is different from the total area of the at least one through hole of the first section and from the total area of the at least one through hole of the second section.

6. The stent of claim 3, wherein the sealant layer is dissolvable when the stent is deployed in a blood vessel.

7. The stent of claim 1, wherein the sealant layer includes
a first section including a plurality of through holes, and
a second section including a plurality of through holes, wherein the first and second sections are of the same size, and wherein the number of through holes of the first section is greater than the number of through holes of the second section.

8. The stent of claim 7, wherein the sealant layer is dissolvable when the stent is deployed in a blood vessel.

9. The stent of claim 1, wherein the sealant layer includes a therapeutic agent, wherein the therapeutic agent of the therapeutic agent layer is different from the therapeutic agent of the sealant layer.

10. The stent of claim 1, wherein the sealant layer includes no therapeutic agent.

11. A stent comprising:
a body;
a first layer of therapeutic agent over at least a section of the body;
a first sealant layer over the first layer of therapeutic agent;
a second layer of therapeutic agent over the first sealant layer; and
a second sealant layer over the second layer of therapeutic agent.

12. The stent of claim 11, further comprising a first through hole that extends through the second sealant layer to reach the second therapeutic agent layer to allow release of the therapeutic agent of the second therapeutic agent layer through the first through hole.

13. The stent of claim 12, further comprising a second through hole that extends through the second sealant layer, the second therapeutic agent layer, and the first sealant layer to reach the first therapeutic agent layer to allow release of the therapeutic agent of the first therapeutic agent layer through the second through hole.

14. The stent of claim 13, further comprising a third through hole that extends through the second sealant layer and the second therapeutic agent layer to reach the first sealant layer.

15. The stent of claim 11, further comprising a through hole that extends through the second sealant layer, the second therapeutic agent layer, and the first sealant layer to reach the first therapeutic agent layer to allow release of the therapeutic agent of the first therapeutic agent layer through the through hole.

16. The stent of claim 11, further comprising:
a first section that includes a first set of at least one hole extending through the second sealant layer to reach the second therapeutic agent layer and a second set of at least one hole extending through the second sealant layer, the second therapeutic agent layer, and the first sealant layer to reach the first therapeutic agent layer; and
a second section that includes a third set of at least one hole extending through the second sealant layer to reach the second therapeutic agent layer and a fourth set of at least one hole extending through the second sealant layer, the second therapeutic agent layer, and the first sealant layer to reach the first therapeutic agent layer.

17. The stent of claim 16, wherein the first and second sections are of the same size, and wherein the ratio of the total area of the first set of at least one hole over the total area of the second set of at least one hole is greater than the ratio of the total area of the third set of at least one hole over the total area of the fourth set of at least one hole.

18. The stent of claim 17, wherein the first section borders an area of blood flow recirculation or stagnation when the stent is deployed in a blood vessel.

19. The stent of claim 17, wherein the second section borders an area of blood flow recirculation or stagnation when the stent is deployed in a blood vessel.

20. The stent of claim 17, wherein the first sealant layer is dissolvable when the stent is deployed in a blood vessel.

21. The stent of claim 17, wherein the second sealant layer is dissolvable when the stent is deployed in a blood vessel.

22. The stent of claim 16, wherein the first and second sections are of the same size, and wherein the ratio of the number of holes in the first set over the number of holes in the second set is greater than the ratio of the number of holes in the third set over the number of holes in the fourth set.

23. The stent of claim 11, wherein the first sealant layer includes a therapeutic agent.

24. The stent of claim 11, wherein the first sealant layer includes no therapeutic agent.

25. The stent of claim 11, wherein the second sealant layer includes a therapeutic agent.

26. The stent of claim 11, wherein the second sealant layer includes no therapeutic agent.

27. A method of fabricating a stent comprising a body, a layer of therapeutic agent over at least a section of the body and a sealant layer over the layer of therapeutic agent, the method comprising:
using a pulsed laser beam to drill a through hole through the sealant layer, wherein the hole allows release of the therapeutic agent of the therapeutic agent layer through the through hole when the stent is deployed in a blood vessel, and wherein the pulsed laser beam has a pulse duration of less than one picosecond.

28. The method of claim 27, wherein a wavelength of the pulsed beam is less than or equal to 800 nm.

29. The method of claim 27, wherein a repetition rate of the laser is 10 to 100 kHz.

30. A method of fabricating a stent comprising a body, a first layer of therapeutic agent over at least a section of the body, a first sealant layer over the first layer of therapeutic agent, a second layer of therapeutic agent over the first sealant layer, and a second sealant layer over the second layer of therapeutic agent, the method comprising:
using a pulsed laser beam to drill a hole through at least one of the second sealant layer, the second layer of therapeutic agent, and the first sealant layer, wherein the pulsed laser beam has a pulse duration of less than one picosecond.

31. The method of claim 30, wherein the hole extends through the second sealant layer to reach the second therapeutic agent layer.

32. The method of claim 31, further comprising using a pulsed laser beam to drill another hole that extends through the second sealant layer, the second therapeutic agent layer, and the first sealant layer to reach the first therapeutic agent layer.

33. The method of claim 30, wherein the hole extends through the second sealant layer and the second therapeutic agent layer to reach the first sealant layer.
